(19) United States
(12) Patent Application Publication
(10) Pub. No.: US 2007/0083086 A1
(43) Pub. Date: Apr. 12, 2007

(54) Shaped retractor blade

(75) Inventors: Steven LeVahn, Lino Lakes, MN (US); Ronald Von Wald, Centerville, MN (US)

Correspondence Address: SHEWCHUK IP SERVICES, 533 77TH STREET WEST, EAGAN, MN, 55121, US

(73) Assignee: LeVahn Intellectual Property Holding Company, LLC, St. Paul, MN (US)

(21) Appl. No.: 11/247,817

(22) Filed: Oct. 11, 2005

(51) Int. Cl. A61B 1/32

(52) U.S. Cl. 600/210; 600/213

(57) ABSTRACT

A shaped surgical retractor blade is used in a surgical retractor assembly. The shaped retractor blade is not formed of sheet material but rather is formed to have a non-uniform thickness between the tissue contacting side and the surgical arena side. The non-uniform thickness can be provided by a longitudinally running rib running between two recesses on surgical arena side of the retractor blade. The recesses provide openings through which the surgeon can better view the surgical arena. The rib provides additional bending strength to the retractor blade. The tissue contacting side of the blade can have a convex curvature, minimizing the possibility of tissue damage at the location that the blade contacts the tissue. The retractor blade can have a constant longitudinally extending section and a distal, tapering section. The uniquely shaped retractor blade can be formed, for instance, such as by injection molding a high tensile strength polymer material which provides desired optical/fluoroscopic/magnetic resonance imaging properties.

### CROSS-REFERENCE TO RELATED APPLICATION(S)

None.

### BACKGROUND OF THE INVENTION

The present invention relates to the field of surgical tools, and particularly to the design and manufacture of surgical retractor systems. Surgical retractor systems are used during surgery to bias and hold tissue in a desired position. As one example, some surgical procedures require anterior access to the spine, through the patient's abdomen. Tissue such as skin, muscle, fatty tissue and interior organs needs to be held retracted to the side so the surgeon can obtain better access to the vertebrae structures of primary interest.

Surgical retraction may be performed by one or more aides using handheld tools, with the most basic retractor apparatus being a tongue depressor. More commonly now in sophisticated operating rooms during abdominal or chest surgery, a surgical retractor system or assembly is used. The retractor assembly may, for instance, include a ring which is rigidly supported from the patient's bed above and around the surgical incision location, with a number of clamps and retractor blades to hold back tissue proximate to the surgical incision. Other retraction systems, such as those disclosed in U.S. Pat. Nos. 6,315,718, 6,368,271 and 6,659,944 to Sharratt, incorporated herein by reference, may not include a ring and/or may be directed at other types of surgery.

Much work has been done to devise better ring and clamping structures for the retractor assemblies. See, for instance, U.S. Pat. No. 4,949,707 and 5,020,195 to LeVahn and LeVahn et al., respectively, and the prior art discussed therein, incorporated herein by reference. Similarly, much work has been done regarding attachment structures to connect the retractor blades to the support ring, post or member. See, for instance, U.S. Pat. No. 4,930,932 to LeVahn, U.S. Pat. No. 5,882,298 to Sharratt and U.S. Pat. Nos. 6,572,540 and 6,602,190 to Dobrovolny, incorporated herein by reference.

Relatively less work has been done in designing the structure of the retractor blades themselves. Most retractor blades are generally flat structures used to press tissue aside, devised after a tongue depressor. In this aspect, the retractor can be considered a “directional-type retractor” because it pulls tissue generally in a single direction away from the surgical arena. Typically, several directional-type retractors are used to pull tissue in different directions away from the wound site. The blades typically attach to a shaft, with the shaft mounted in a generally horizontal orientation and extending radially outward above the surgical arena. Typical blades include a sweeping approximately right angle transverse bend so the blade portion is directed downward into the surgical incision. Different lengths and widths of retractor blades are commonly provided to the surgeon, but the vast majority of retractor blades are cut and bent structures formed from sheets of surgical stainless steel. While various more exotic blade structures have been devised to give the blade some flexibility to change in shape or size during surgery (see, for instance, U.S. Pat. Nos. 1,947,649, 3,749,088, 4,190,042, 5,080,088 and 5,722,935), the vast majority of retractor blades remain generally rigid structures which do not change shape.

Other types of devices which may be considered “lumen-type retractors” are designed to surround the surgical arena or area of interest, using a compressive hoop stress to hold the tissue back in all directions (or at least substantially equal and opposite directions) simultaneously. The present invention, though having aspects which can also be applied to lumen-type retractors, is primarily directed at blades for directional-type retractors rather than for lumen-type retractors.

Surgical retractor systems should facilitate the goal of having the smallest possible incision while still permitting the surgeon unobstructed access when performing the surgical technique. In general, smaller incisions reduce discomfort to the patient, decrease recovery time, and decrease the amount of scarring from the surgery. Surgical retractor systems must be robust and strong, as even a slight possibility of failure during use is not tolerated. Surgical retractor assemblies should be readily reusable, including sterilizable, for use in multiple surgeries. Surgical retractor assemblies should be designed for proper surgical imaging results, including fluoroscopy and magnetic resonance imaging (“MRI”). Surgical retractor systems should maintain a relatively low cost. Improvements in surgical retractor blades can be made in keeping with these goals.

### BRIEF SUMMARY OF THE INVENTION

The present invention is a shaped surgical retractor blade, and a retractor assembly using the shape retractor blade. In contrast to prior art retractor blades, the shaped retractor blade is not formed of sheet material but rather has a non-uniform thickness between the tissue contacting side and the surgical arena side. In one aspect, the tissue contacting side of the blade has a convex curvature, and the non-uniform thickness is provided by a longitudinally running rib on the retractor blade. In another aspect, the blade has at least one recess running longitudinally at an intermediate position on the surgical arena side between the edges. The uniquely shaped retractor blade can be formed, for instance, such as by injection molding a high tensile strength polymer material which provides desired optical/fluoroscopic/magnetic resonance imaging properties.

### BRIEF DESCRIPTION OF THE DRAWINGS

FIG. 1 is a perspective view of a preferred shaped retractor blade in accordance with the present invention.

FIG. 2 is a plan view of the retractor blade of FIG. 1.

FIG. 3 is a cross-sectional view of the retractor blade of FIGS. 1-2 taken along lines 3-3.

FIG. 4 is a cross-sectional view of the retractor blade of FIGS. 1-3 taken along lines 4-4.

FIG. 5 is a perspective view of the retractor blade of FIGS. 1-4 as used in a surgical retractor assembly.

FIG. 6 is a cross-sectional view similar to FIG. 4 of a first alternative retractor blade in accordance with some aspects of the invention.

FIG. 7 is a cross-sectional view similar to FIG. 4 of a second alternative retractor blade in accordance with some aspects of the invention.

FIG. 8 is a cross-sectional view similar to FIG. 4 of a third alternative retractor blade in accordance with some aspects of the invention.

FIG. 9 is a cross-sectional view similar to FIG. 4 of a fourth alternative retractor blade in accordance with some aspects of the invention.

FIG. 10 is a cross-sectional view similar to FIG. 4 of a fifth alternative retractor blade in accordance with some aspects of the invention.

FIG. 11 is a cross-sectional view similar to FIG. 4 of a sixth alternative retractor blade in accordance with some aspects of the invention.

FIG. 12 is a cross-sectional view similar to FIG. 4 of a seventh alternative retractor blade in accordance with some aspects of the invention.

FIG. 13 is a cross-sectional view similar to FIG. 4 of a eighth alternative retractor blade in accordance with some aspects of the invention.

FIG. 14 is a cross-sectional view similar to FIG. 4 of a ninth alternative retractor blade in accordance with some aspects of the invention.

FIG. 15 is a cross-sectional view similar to FIG. 4 of a tenth alternative retractor blade in accordance with some aspects of the invention.

FIG. 16 is a cross-sectional view similar to FIG. 4 of a eleventh alternative retractor blade in accordance with some aspects of the invention.

FIG. 17 is a cross-sectional view similar to FIG. 4 of a twelfth alternative retractor blade in accordance with some aspects of the invention.

While the above-identified drawing figures set forth one or more preferred embodiments, other embodiments of the present invention are also contemplated, some of which are noted in the discussion. In all cases, this disclosure presents the illustrated embodiments of the present invention by way of representation and not limitation. Numerous other minor modifications and embodiments can be devised by those skilled in the art which fall within the scope and spirit of the principles of this invention.

### DETAILED DESCRIPTION

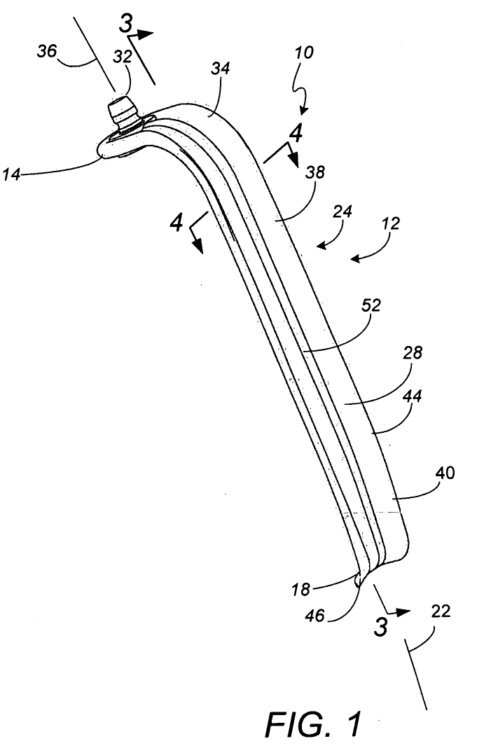

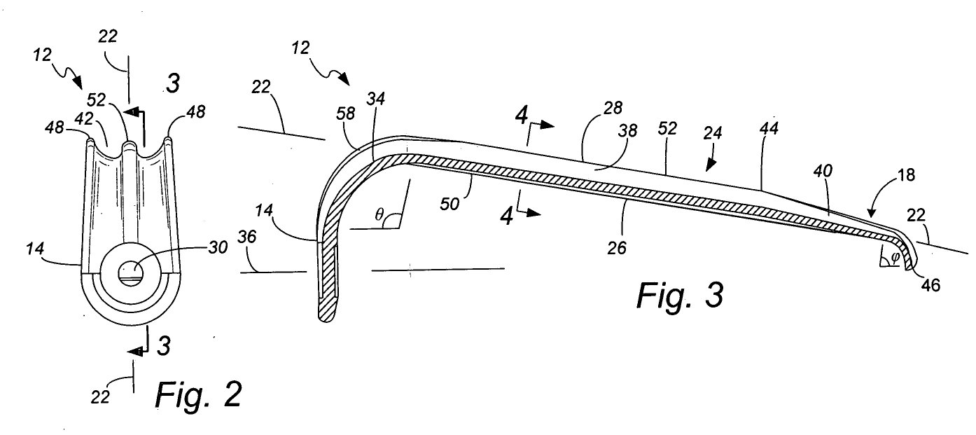

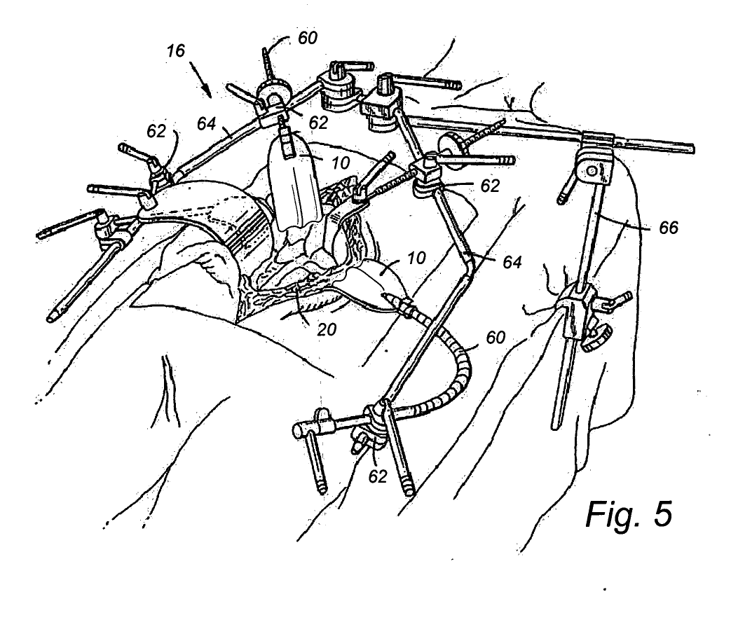

A first embodiment of a shaped surgical retractor blade 10 in accordance with the present invention is shown in FIGS. 1-4. This particular embodiment is a renal vein blade 10, having a blade width of about ⅞^{th} inch and a blade length of about six inches. The blade 10 has a blade body 12 with a proximal end 14 for attachment as part of a retractor assembly 16 (shown further in FIG. 5). The blade body 12 has an opposing distal end 18 which extends into the surgical incision 20 (as shown in FIG. 5). In the preferred embodiment, the retractor blade 10 is symmetrical about a longitudinal bisecting plane which includes a longitudinal axis 22. Between the proximal and distal ends 14, 18, the blade body 12 includes an extending portion 24. The extending portion 24 for the preferred renal vein blade 10 extends generally linearly when depicted in longitudinal cross-section as shown in FIG. 3, providing the length of the blade 10. It is noted that the invention can be equally applied to other shapes and sizes (see, e.g., FIG. 5) as well as non-symmetrical profiles, for use such as in other particular types of surgeries. The blade body 12 has a tissue contacting side 26 and an opposing surgical arena side 28. In use, the distal end 18 of the blade 10 is pushed generally vertically downward into the abdominal incision 20, and then the blade 10 is pulled generally horizontally in the direction of the tissue contacting side 26, so the tissue contacting side 26 presses on tissue and retracts tissue from the surgical arena.

The proximal end 14 of the blade body 12 preferably includes an opening 30, with a connection pin 32 extending through the opening 30 and attached to the blade body 12. For example, the connection pin 32 may be as disclosed in U.S. Pat. No. 5,882,298 to Sharratt, incorporated herein by reference. Alternatively, the proximal end 14 of the blade body 12 may permit attachment to the rest of the retractor assembly 16 by other means.

Not far from the attachment opening 30, the blade body 12 sweeps in a wide transverse bend 34. In the preferred embodiment of a six inch renal vein blade 10, the transverse bend 34 is provided as a circular arc having a inside diameter of about 1¼ inches. The preferred transverse bend 34 extends in an arc θ of about 97°. If the axis 36 of the connection pin 32 is taken as vertical, the 97° arc θ results in the extending portion 24 of the blade 10 having a slight (7°) retrograde slant, i.e., such that in use the distal end 18 of the blade 10 pulls tissue back slightly (about ¾ inch) further than the proximal end of the extending portion 24. The slight retrograde slant is particular appropriate for abdominal use, wherein the musculature of the abdominal wall is both tighter (i.e. more difficult to retract) and stiffer (i.e., once retracted in one location, less likely to flex and flow between adjacent retraction blades 10 into the surgical arena) than the underlying organs to be retracted such as intestines.

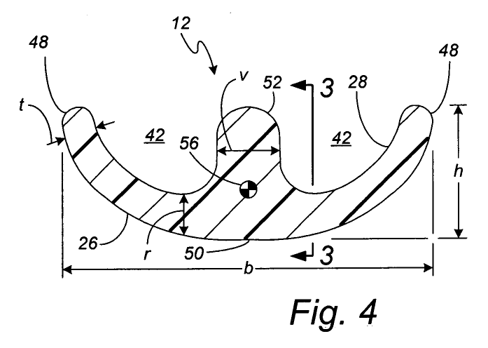

As best shown in FIG. 3, the extending portion 24 includes a first generally constant longitudinal section 38 and a second, tapering longitudinal section 40. In the preferred six inch renal vein blade 10, the constant section 38 extends for about four inches from the transverse bend 34, with the tapering section 40 occupying about the final 1½ inches of length of the blade 10. The preferred material of constant thickness section 38 has a root thickness r, measured in the y-direction and at the bottom of the recess 42 as shown in FIG. 4, of about 1/10th of an inch thick. Beginning at a break-in-slope point 44, the root thickness r is decreased to a minimum thickness of about 1/24^{th} of an inch, i.e., less than half of the root thickness of the constant section 38.

The distinction between the constant section 38 and the tapering section 40 as denoted by the break-in-slope point 44 provides several advantages. Firstly, during retraction, the different portions of longitudinal extent of the blade 10 support different stress forces and different bending moments. Even if all of the horizontal retraction force of the tissue is placed only at the distal end 18 of the blade 10, the local bending moments will necessarily be less toward the distal end 18 than toward the transverse bend 34, because the horizontal retraction force acts through a shorter moment arm. Having the need to support less bending stress, the tapering longitudinal section 40 need not be as thick and strong against bending as the first section 38.

Secondly, it is recognized that a wide variety of different surgical procedures are performed on a wide variety of patients having differing tissue shapes and strengths, that is, that each surgery is unique and each patient's anatomy is unique. The blade 10 should be designed to be most useful in the greatest number of unique surgeries and in the greatest number of unique anatomies which might be encountered. Because of the unique surgeries and unique anatomies, the blade 10 is positioned at different heights and different orientations in each surgery. Indeed, the flexibility of the surgeon in positioning the retractor structure 16 is the primary reason that so much work has been done in designing the post, ring, support and clamping structures of the prior art and why post, ring, support and clamping improvements will continue, all of which may be used with the present invention. However, the distinction between the constant section 38 and the tapering section 40, with a break-in-slope point 44 visible upon inspection of the blade 10, almost subconsciously influences the surgeon's decision of where to position the blade 10 relative to the tissue structure. In most instances the surgeon will intuitively position the blade 10 at least deeply enough that the tighter, stiffer muscle tissue contacts the constant section 38 rather than the tapering section 40. Such placement ensures that the moment arm of the primary retracting force about the transverse bend 34 is no longer than the constant section 38, i.e., less than about 4½ inches. By influencing the surgeon to position the blade 10 at a desired height relative to the height of the abdominal muscle, the blade 10 itself minimizes the amount of bending stress to which it will be subjected during use. Less blade bending during use is beneficial in numerous respects, including less creep (change in deflection) of the blade 10 during surgery, and including less possibility of blade breakage or other failure and as importantly less perceived possibility of blade breakage or other failure, etc. By having a both a tapering section 40 and a constant section 38 as part of the extending section of the blade body 12, the blade 10 is more consistently positioned in more surgeries and achieves a more satisfactory outcome.

Distal to the tapering section 40 of the extending portion 24 of the blade body 12, the blade 10 terminates in a hook portion 46. In the preferred six inch renal vein blade 10, the hook section 46 is provided by a sweeping transverse bend in a circular arc φ of about 83° having an inside diameter of about ½ inch. In the hook section 46, the root thickness of the material is increased beyond the root thickness of the tapering section 40 to, in the preferred embodiment, a root thickness r of about 1/14^{th} inch. That is, the hook section 46 has a root thickness which is greater than the minimum root thickness of the tapering section 40 but less than the root thickness of the constant section 38. Similarly, the hook section 46 has a standard thickness which is greater than the minimum standard thickness of the tapering section 40 but less than the standard thickness of the constant section 38. In the preferred six inch renal vein blade 10, the standard thickness t of the hook section is about 1/14^{th} of an inch. The hook section 46 of the blade body 12 serves primary importance during insertion of the blade 10 into the incision 20 and downward through tissue. The standard thickness of the hook section 46 is selected to be appropriately blunt to minimize damage to tissue during insertion, in contrast to a knife edge which would result if the tapering section 40 were carried to its full extreme.

The preferred blade 10 also has significant shape characteristics which run longitudinally, best shown with reference to FIG. 4. The blade body 12 includes first and second longitudinally extending edges 48. In the preferred renal vein blade 10, these first and second edges 48 are positioned significantly above a base 50 of the tissue contacting side 26. That is, the tissue contacting side 26 depresses tissue at the base 50 significantly further than it depresses tissue at its first and second edges 48. The difference in height between the base 50 and the first and second edges 48 is preferably achieved by a relatively smooth convex curvature shown in FIG. 4. This curvature is selected to best match the tear strength of the tissue to which the blade 10 is likely to be used. In other words, the curvature of the tissue contacting side 26 is intended to permit the greatest access to the surgical arena with the smallest incision and the least possible tissue damage. In the preferred embodiment, the curvature of the tissue contacting side 26 is provided in a nearly elliptical arc providing the ⅞^{th} inch width of the blade 10. The edges 48 of the blade body 12 are generously radiused so as to minimally damage tissue and provide an aesthetically pleasing appearance. In the preferred embodiment, the edges 48 of the blade body 12 are radiused the full standard thickness of the blade body 12. That is, in the constant section 38 of the blade 10, the edges 48 of the preferred renal vein blade 10 are provided with a radius of about 1/24^{th} of an inch (i.e., the greatest radius permitted by the 1/12^{th} inch standard thickness t). This radius of each edge 48 tapers over the tapering portion to a radius of about 1/48^{th} of an inch (i.e., the greatest radius permitted by the 1/24^{th} inch standard thickness t).

A central rib 52 is provided running along the longitudinal bisecting plane of the blade 10. The rib 52 defines two recesses 42 each running longitudinally between the rib 52 and one of the edges 48. The recesses 42 are important in providing a field of view to the surgeon to the surgical arena which is not overly impinged upon by the retractor blade 10. Each recess 42 has a depth which is preferably at least 10% of the retractor blade width. Once the bottom of each recess 42 is reached, the blade body 12 extends transversely outward from the longitudinal axis 22 with a region of generally constant thickness t between the tissue contacting side and the surgical arena side. For the preferred six inch renal vein blade 10, the thickness t as measured normal to the tissue contacting side and the surgical arena side is about 1/12^{th} of an inch, and the recesses 42 extend just more than ⅙^{th} of an inch below the edges 48.

The rib 52 provides significant additional bending strength to the blade body 12 which could otherwise not be achieved by a rectangular cross-section blade body 12 of the standard thickness t or a rectangular cross-section blade of the same cross-sectional area. The rib 52 preferably extends around the transverse bend 34, but need not extend into the hook section 46. In the preferred embodiment, the rib 52 has a height from the base 50 which is, in the constant section 38, equal to the height of the edges 48 from the base 50 (both marked as height h in FIG. 4). In the dimensions of the preferred renal vein blade 10, the rib 52 provides an additional thickness of almost ¼^{th} of an inch beyond the 1/12^{th} inch standard thickness t (the recesses 42 are over ⅙^{th} inch deep), for a total thickness h at the rib 52 of about 5/16^{th} of an inch. To provide the additional bending strength, the rib preferably has a rib width v which is from one third to three times as great as the generally constant thickness t of the transversely extending region. The rib 52 more preferably has a width v at least equal to the standard thickness t of the constant section 38, and most preferably about 1½ the root thickness r. For instance, the preferred rib 52 has a width v of about 0.15 inches. The preferred rib 52 is rounded at its top as shown in the cross-section of FIG. 4, providing an aesthetic appearance with no sharp edges and further minimizing interference with the surgeon's view into the surgical arena. The height of the rib 52 tapers in the tapering section 40, equivalent to the taper in the root thickness. That is, in the preferred renal vein blade 10 as the root thickness r tapers to about 1/24^{th} of an inch, the rib height tapers to a height of about 1/12^{th} of an inch, for a total minimum thickness h at this rib 52 of about ⅛^{th} of an inch.

In addition to the benefits of minimum tissue damage associated with the convex curvature of the tissue contacting side 26 of the blade 10, the height of the edges 48 and the rib thickness provide significant bending strength benefits to the blade 10. These bending strength benefits can be best analyzed through applying beam theory to the transverse cross-section of the blade body 12 shown in FIG. 4. By considering the blade body 12 as a beam, the blade 10 can be modeled as having a centroid 56 and as having a moment of inertia (second moment of area) as known in beam theory arts. In particular, the blade 10 can be modeled as having its moment of inertia defined as
Iₓₓ=∫y²∂A
at any given transverse cross-section, and bending strength is proportional to moment of inertia. For the cross-section shown in FIG. 4, with a width b of about ⅞^{th} of an inch, a root thickness r of about 1/10^{th} of an inch, and a blade cross-sectional height h of about 5/16^{th} of an inch, the blade body profile has a cross-sectional area of about 12.6×10⁻² in² and a moment of inertia of approximately 10.9×10⁻⁴ in⁴. The moment of inertia of the blade body profile can then be compared against the moment of inertia of a rectangular cross-section, either with the same cross-sectional area or with a thickness equal to the height h of the blade body 12. As is well known in the beam theory art, the moment of inertia of a rectangular beam is
Iₓₓ=bh³/12
The moment of inertia of a rectangular beam with a width b of ⅞^{th} inch and a cross-sectional area of 12.6×10⁻² in² (i.e., a thickness h of about 1/7^{th} inch) is about 2.2×10⁻⁴ in⁴. That is, the retractor blade 10 of the preferred cross-section shown in FIG. 4 is about 5 times as strong in bending strength as compared to a blade of the same mass and length but with a rectangular cross-section. The cross sectional area of a rectangular beam with a width b of ⅞^{th} inch and a thickness h of 5/16^{th} inch is about 0.27 in², and the moment of inertia of such a rectangular beam is about 22.3×10⁻⁴ in⁴. That is, the retractor blade 10 of the preferred cross-section shown in FIG. 4 has a cross-sectional area which is less than half that of a rectangular beam with the same height and width, i.e., less than bh/2, and a moment of inertia which is less than half that of the rectangular beam, i.e, less than bh³/24.

At the same time, were bending strength the only parameter to be maximized, the blade body 12 would take on the cross-sectional shape typical of building structural members, e.g. rectangular I-beams. Instead, the preferred embodiment of the present invention achieves a significant bending strength with a minimal amount of material, while still maintaining the counterveiling benefits of providing a minimal visual disturbance into the surgical arena and providing a smooth convex contact surface for minimal tissue damage.

Bending strength of the blade 10 is most significant at the transverse bend 34. To achieve yet further increases in bending stiffness, while not overly impacting upon the surgeon's view into the surgical arena, the edges 48 are raised even further from the base 50 of the blade body 12. This is best shown in FIG. 3, wherein the raised edge 58 can be seen at the transverse bend 34 extending further than the rib 52. To minimize the interference of the rib 52 into the surgeon's viewpath, the rib 52 is not similarly extended at the transverse bend 34, but rather retains the same height (in the preferred embodiment, about ⅕^{th} of an inch above the base 50).

It will be readily understood that the preferred retractor blade 10 of the present invention can no longer be formed merely by bending sheet material, because the tissue contacting side 26 of the blade body 12 is not uniformly spaced from the surgical arena side 28. If desired for maximum strength, durability and repeated use, the present invention could be formed by machining surgical steel into the shaped profile shown. In fact, the dimensions of the preferred blade 10 as described herein, if machined out of surgical grade stainless steel, would result in an extremely stiff, rigid and heavy blade 10. Instead the preferred embodiment is formed of a surgical grade polymer. The preferred method of forming the polymer material into the shaped retractor blade body 12 shown is through injection molding, which produces high quality consistent parts at a minimal cost. It might be possible to alternatively obtain many or all features of the preferred embodiment through an extrusion process, followed by malleable bending and/or machining.

By forming the shaped retractor blade body 12 out of polymer, the blade body 12 can be formed to be largely or entirely radiolucent, thereby achieving better fluoroscopic imaging results for the surgeon. The blade body 12 can also be formed of a material with minimal magnetic susceptibility as disclosed in U.S. Pat. No. 5,882,298 to Sharratt, thereby achieving better MRI imaging results. If desired, the polymer selected can be a transparent or translucent material after molding, permitting the surgeon to see through the blade body 12 during surgery.

For instance, the polymer material could be acrylic, acetal, nylon, polyester (PBT, PET), PTFE, PVC, polycarbonate, rigid thermoplastic urethane (RTPU), polyethylene, polypropylene, ABS, polysulfone, polyethersulfone, polyphenylsulfone, polyetherimide, polyetherketone or similar polymer materials. All such materials are considered generally rigid at body temperature, meaning that the material does not lose its shape during surgery and will by itself support the retraction load applied to the retractor blade 10, possibly with deflection. These materials could be used with or without additives such as carbon fibers or glass beads to increase strength or rigidity. The material selected should have a high tensile strength, and preferably can withstand repeated use including sterilization by all common techniques (including electron-beam radiation, gamma radiation, autoclaving and ETO sterilization). As most preferred materials, ULTEM 1000 polyetherimide (GE Plastics) and PEEK polyetherketone (Victrex USA, Inc., Greenville, S.C.) perform nicely.

FIG. 5 depicts the use of multiple retractor blades 10 in a retractor blade assembly 16, which also may include prior art retractor blades. Each retractor blade 10 is attached to a retractor blade shaft 60. The connection with the retractor blade shaft 60 permits the retractor blade 10 to pivot slightly about the generally vertical axis 36 defined by the connection pin 32 (shown in FIG. 1) or other structure used to attach the retractor blade body 12 to its shaft 60. Each retractor blade shaft 60 is clamped with a clamp 62 to a retractor ring 64. The clamps 62 permit the surgeon to adjust the horizontal location (in and out) as well as the angular orientation of the shafts 60, and then permit the surgeon to securely fasten the retractor blade 10 once a desired position and orientation is achieved. The retractor ring 64 is supported relative to the patient's bed, such as by clamping to one or more retractor posts 66. Each of the retractor blade shaft 60, the clamp 62, the retractor ring 64 and the retractor post 66 may be formed primarily of a metal material such as surgical stainless steel as known in the surgical retractor art. If desired, the retractor blade shaft 60 could alternatively be made integral with the retractor blade 10 or integral with the retractor ring 64.

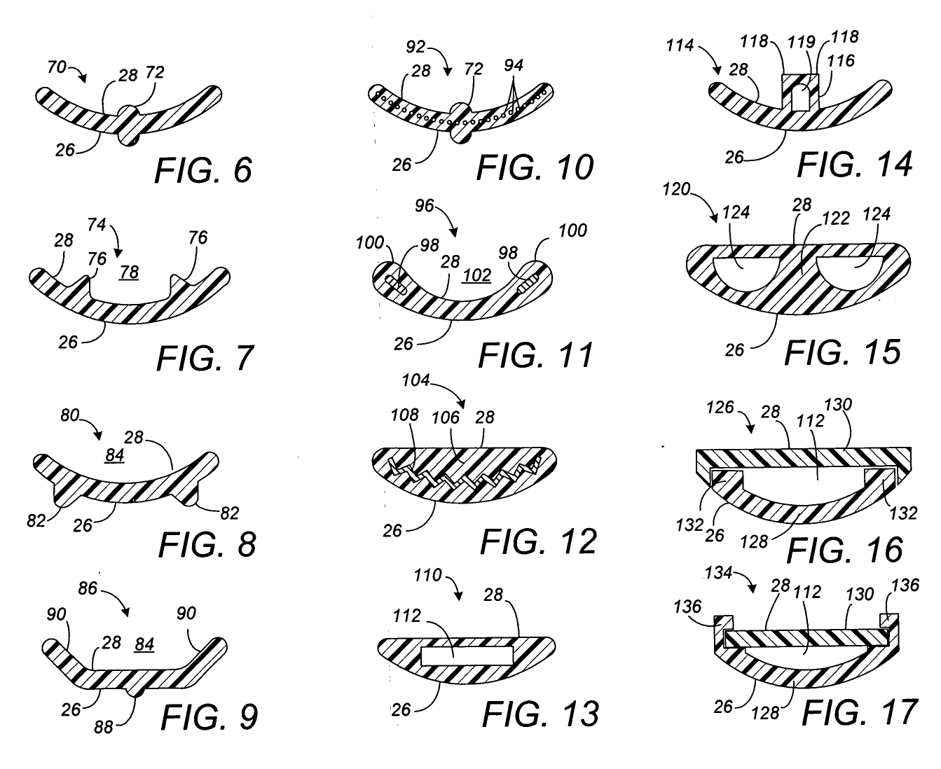

FIG. 6 shows a first alternative embodiment of a retractor blade 70 in accordance with the present invention. The retractor blade 70 is much like the retractor blade 10 previously described, but the central rib 72 has been moved lower relative to the longitudinal axis of the retractor blade 70 than the central rib 52 of the retractor blade 10. With the central rib 72 moved lower, the rib 72 extends from both the surgical arena side 28 and the tissue contacting side 26 of the blade 70. With the central rib 72 not extending quite as high, the retractor blade 70 gives the surgeon even better view into the surgical arena. As part of moving the rib 72 lower toward the tissue contacting side 26 of the blade 70, the blade 70 is not quite as convex on the tissue contacting side 26. With a shallower curve on the tissue contacting side 26, blade 70 still retains roughly the same overall thickness or height h as blade 10.

FIG. 7 shows a second alternative embodiment of a retractor blade 74. The retractor blade 74 is much like the blades 10, 70, but the rib is split into two ribs 76 on the surgical arena side 28 of the blade 74. The two ribs 76 leave the retractor symmetrical about its bisecting plane, but open a wider central recess 78 on the surgical arena side 28. Further, instead of having the fully rounded shape of ribs 52 and 72, the ribs 76 are more triangular shaped. The more triangular shape is appropriate on the surgical arena side 28, as the ribs 76 will not contact tissue during retraction and thus will not contribute to any tearing or other injury to tissue during the surgery. Still, the tips of the triangular ribs 76 are rounded, so as to avoid sharp edges in manufacture and handling.

FIG. 8 shows a third alternative embodiment of a retractor blade 80. Retractor blade 80 is similar to retractor blade 74, but the ribs 82 are on the tissue contacting side 26 rather than on the surgical arena side 28. Because the ribs 82 are on the tissue contacting side 26, the curvature on the tips of the triangular ribs 82 is made with a greater radius, thereby minimizing damage to tissue during retraction. With the ribs 82 entirely on the tissue contacting side 26, the central recess 84 is even larger permitting view into the surgical arena.

FIG. 9 shows a fourth alternative embodiment of a retractor blade 86. Retractor blade 86 can be thought of as a combination between retractor blade 70 of FIG. 6 and retractor blade 80 of FIG. 8. A single rib 88 is provided which extends longitudinally only on the tissue contacting side 26. With the single rib 88 on the tissue contacting side 26 and located on the base, care must be taken not to extend the rib 88 too far or too sharply from the tissue contacting side 26 so as to create a potential knife edge or tissue damaging structure. Additionally, the blade 86 has angled wing portions 90 rather than merely a circular convex curvature. The angled wing portions 90 provide the same benefits as the convex curvature of the other embodiments, but may be easier to machine in the injection mold or otherwise lead to a more simply manufactured blade 86. For all of the embodiments disclosed, the convex curvature of the tissue contacting side 26 could be achieved with angled wing portions.

FIG. 10 shows a fifth alternative embodiment of a retractor blade 92. The blade 92 has an outside shape which is identical to outside shape of the blade 70 of FIG. 6. Inside the cross-section, however, the polymer material of the blade 90 is reinforced with longitudinally running fibers 94. The longitudinally running fibers 94 particularly assist in increasing the strength and durability of the blade 92 (similar to rebar in concrete structures), preventing any possibility of the blade 92 from cracking due to local tensile stress under use.

FIG. 11 shows a sixth alternative embodiment of a retractor blade 96. The blade 96 obtains its additional strength not from a central rib but rather from two longitudinally running stiffening rods 98, coupled within wing portions 100 of greater thickness. The stiffening rods 98 can, for instance, be metallic inserts in the injection molded body of the retractor blade 96. By being located in the wing portions 100, the shape of the blade 96 still leaves a large recess 102 on its surgical arena side 28.

FIG. 12 shows a seventh alternative embodiment of a retractor blade 104. While blade 104 still has the convex shape provided to its issue contacting side 26, it foregoes the advantages associated with providing a recess on its surgical arena side 28. The additional strength is provided by having the center section 106 of the blade 104 be of greater thickness. At the same time, blade 104 replaces the fibers 94 of the blade 92 of FIG. 10 and the stiffening rods 98 of the blade 96 of FIG. 11 with a corrugated core 108. The core 108 can, for instance, be a metallic insert in the injection molded body of the retractor blade 96. By having the core 108 corregated with longitudinally running fold locations, the core 108 can provide significant additional bending strength to the blade 104.

FIG. 13 shows an eighth alternative embodiment of a retractor blade 110. Retractor blade 110 has an exterior shape which is substantially identical to the exterior shape of retractor blade 104 of FIG. 12. In this case, the blade 110 is hollowed out with a cavity 112. Cavity 112 causes the blade 110 to be lighter. At the same time, by having the cavity 112 located in the center of the blade 110, the material lost due to the cavity 112 causes only an insignificant loss in bending strength (moment of inertia) of the blade 110. In addition to injection molding techniques, blade 110 in particular can be formed by extruding a circular lumen of polymer material, and then rolling the heated polymer material to form the generally flattened shape shown, which would result in a cavity 112 of only slightly modified cross-sectional shape.

FIG. 14 shows a ninth alternative embodiment of a retractor blade 114. Retractor blade 114 has an overall shape, including a central rib 116, similar to the overall shape and the central rib 52 of the blade 10 of FIGS. 1-4. In contrast to the rib 52, the central rib 116 is permitted to have sharp corners 118. Such sharp corners 118 are permissible on the surgical arena side 28 where they do not make damaging contact with the patient's tissue. Similar to the cavity 112 of the blade 110 of FIG. 13, blade 114 has a central cavity 119 disposed within the rib 116. The central cavity 119 lightens the blade 114, while causing only an insignificant loss in bending strength (moment of inertia) of the blade 110.

FIG. 15 shows a tenth alternative embodiment of a retractor blade 120. The retractor blade 120 can be thought of as a combination between the retractor blade 10 of FIGS. 1-4 and the retractor blade 110 of FIG. 13. That is, the retractor blade 120 still has a central rib 122, but recesses 42 are replaced by cavities 124. The retractor blade 120 sacrifices the viewing into the surgical arena provided by recesses 42 to obtain additional strength and a smooth outer profile. At the same time, the cavities 124 lighten the blade 120, while causing only an insignificant loss in bending strength (moment of inertia) of the blade 120.

FIG. 16 shows an eleventh alternative embodiment of a retractor blade 126. Retractor blade 126 is similar to the retractor blade 110 of FIG. 13, but permits a different method of manufacture. In particular, retractor blade 126 is formed in two parts, a tissue contacting bottom 128 and a surgical arena top 130. The tissue contacting bottom 128 and the surgical arena top 130 can be formed by various simple methods, including injection molding, extruding, etc., without the difficulty of forming a cavity. After separate formation, the tissue contacting bottom 128 and the surgical arena top 130 can be joined together, such as through adhesives, sonic welding, etc. The primary additional strength of blade 126 is provided by the thick wing portions 132 and by a good overall moment of inertia.

FIG. 17 shows a twelfth alternative embodiment of a retractor blade 134. The blade 134 is similar to the retractor blade 126 of FIG. 16, but the wing portions 136 extend up and over the surgical arena top 130. By having this profile, the wing portions 136 positively engage the surgical arena top 130 and prevent any possibility of the surgical arena top 130 becoming separated from the tissue contacting bottom 128 during flexing of the blade 134 during retraction.

As will be understood, all of these embodiments provide advantages over the prior art. The increase in bending strength provided due to the varying thickness profile of all these embodiments facilitate their manufacture by polymer, rather than solely metallic, materials. None of the embodiments are merely sheet material constructions, but rather have a characteristic profile which makes each embodiment particularly suitable for use as a directional retractor.

Although the present invention has been described with reference to preferred embodiments, workers skilled in the art will recognize that changes may be made in form and detail without departing from the spirit and scope of the invention. For instance, while all drawings show the embodiments in the preferred polymer material, all the embodiments would retain their shape advantages even if formed of metal.

1. A surgical retractor blade assembly comprising:
a shaft for supporting the surgical retractor blade assembly from a surgical support, the shaft having a shaft axis; and
a retractor blade connectable to the shaft such the retractor blade extends at an angle relative to the shaft axis, the retractor blade being unitarily formed of a generally rigid material, the retractor blade having a tissue contacting side and a surgical arena side opposing the tissue contacting side, wherein the thickness of the retractor blade between the tissue contacting side and the surgical arena side is not uniform.

2. The surgical retractor blade assembly of claim 1, wherein the retractor blade is formed of a non-metallic polymer material.

3. The surgical retractor blade assembly of claim 2, wherein the retractor blade is injection molded.

4. The surgical retractor blade assembly of claim 2, wherein the shaft is formed of metal.

5. The surgical retractor blade of claim 1, wherein the non-uniform thickness is created by a longitudinally extending rib.

6. The surgical retractor blade of claim 5, wherein the retractor blade has at least one transversely extending region of generally constant thickness defined between the tissue contacting side and the surgical arena side, wherein the rib has a rib thickness which is at least as great as the generally constant thickness of the transversely extending region.

7. The surgical retractor blade of claim 6, wherein the rib has a rib width which is from one third to three times as great as the generally constant thickness of the transversely extending region.

8. The surgical retractor blade assembly of claim 1, wherein the retractor blade comprises a transverse bend from a shaft attachment proximal end to a tissue contacting area, such that the tissue contacting area extends at an angle relative to the shaft attachment proximal end.

9. The surgical retractor blade assembly of claim 8, wherein the non-uniform thickness is created by a longitudinally extending rib which extends around the transverse bend.

10. The surgical retractor blade assembly of claim 1, wherein the non-uniform thickness is created by a longitudinally extending tapering section.

11. The surgical retractor blade assembly of claim 10, wherein the blade comprises an extending portion which includes a longitudinally extending constant section and a longitudinally extending tapering section.

12. The surgical retractor blade assembly of claim 1, wherein a transverse cross-section of the retractor blade has a thickness h and a width b, wherein the retractor blade has a moment of inertia about a widthwise axis 22 at the centroid of that cross section which is less than bh³/24.

13. A surgical retractor blade for a directional retractor comprising:
a blade portion running longitudinally from a proximal connection end to an opposing distal end, the blade portion having a tissue contacting side and a surgical arena side opposing the tissue contacting side, the blade portion defining a longitudinal axis, wherein the blade portion comprises:
a first edge running longitudinally on one side of the longitudinal axis;
a second edge running longitudinally on an opposing side of the longitudinal axis to the first edge; and
at least one portion of increased thickness running longitudinally between the first edge and the second edge;
wherein the tissue contacting side has a generally convex curvature so as to retract tissue further at a base of the blade than at the first edge and the second edge.

14. The surgical retractor blade of claim 13, wherein the retractor blade is unitarily formed of a non-metallic polymer material.

15. The surgical retractor blade of claim 13, wherein the portion of increased thickness is provides by at least one rib running longitudinally at an intermediate position between the first edge and the second edge.

16. The surgical retractor blade of claim 15, wherein the surgical retractor blade further comprises:
a shaft attachment end portion; and
a transverse bend portion connecting the shaft attachment end portion to the blade portion, such that the blade portion extends at an angle relative to the shaft attachment end portion, wherein the rib extends around the transverse bend portion.

17. A surgical retractor blade for a directional retractor comprising:
a blade portion running longitudinally from a proximal connection end to an opposing distal end, the blade portion having a tissue contacting side and a surgical arena side opposing the tissue contacting side, the blade portion defining a longitudinal axis, wherein the blade portion comprises:
a first edge running longitudinally on one side of the longitudinal axis;
a second edge running longitudinally on an opposing side of the longitudinal axis to the first edge, with a retractor blade width between the first edge and the second edge; and
at least one recess running longitudinally at an intermediate position between the first edge and the second edge, the recess being defined on the surgical arena side, the recess having a depth which is at least 10% of the retractor blade width.

18. The retractor blade of claim 17, further comprising a rib running longitudinally between the first edge and the second edge, wherein the tissue contacting side has a generally convex curvature so as to retract tissue further at a base of the rib than at the first edge and the second edge.

19. The retractor blade of claim 18, wherein the rib raises above the depth of the recess by a distance which is at least 10% of the retractor blade width.

20. The surgical retractor blade of claim 17, wherein the retractor blade is unitarily formed of a non-metallic polymer material.
